(19) United States
(12) Patent Application Publication
(10) Pub. No.: US 2023/0077209 A1
(43) Pub. Date: Mar. 9, 2023

(54) Bacteriophage Cocktail-Containing Hydrogel Compositions and Methods of Production and Use Thereof

(71) Applicant: William Colton, Bellaire, TX (US)

(72) Inventor: William Colton, Bellaire, TX (US)

(21) Appl. No.: 17/799,796

(22) PCT Filed: Feb. 15, 2021
(86) PCT No.: PCT/US2021/018113

§ 371 Date: Aug. 15, 2022

Related U.S. Application Data
(60) Provisional application No. 62/976,663, filed on Feb. 14, 2020.

(51) Int. Cl. A61L 29/16; A61L 29/08; A61L 29/04; A61L 29/14; A61L 24/00; A61L 24/04; A61K 35/76

(52) U.S. Cl. CPC A61L 29/16; A61L 29/085; A61L 29/049; A61L 29/145; A61L 24/0031; A61L 24/043; A61L 24/0015; A61K 35/76; A61L 2300/30; A61L 2300/602; A61L 2300/404; A61L 2420/02; A61L 2400/04; A61L 2420/04; A61L 2420/06

(57) ABSTRACT

An anti-bacterial coating composition for use with a medical implant is disclosed. The anti-bacterial coating composition includes a bacteriophage cocktail that is encapsulated in beads that are embedded within a hydrogel. Also disclosed are kits containing the anti-bacterial coating composition as well as methods of producing and using the coating composition.

### CROSS REFERENCE TO RELATED APPLICATIONS/INCORPORATION BY REFERENCE

This application claims benefit under 35 USC § 119(e) and PCT Rule 80.5 of U.S. Provisional Application No. 62/976,663, filed Feb. 14, 2020. The entire contents of the above-referenced patent application(s) are hereby expressly incorporated herein by reference.

### STATEMENT REGARDING FEDERALLY SPONSORED RESEARCH OR DEVELOPMENT

Not Applicable.

### BACKGROUND

Indwelling medical devices (such as, but not limited to, urinary catheters) are frequently colonized with microorganisms, resulting in the formation of microbial biofilms on and around the devices. Biofilms possess a complex matrix of extracellular polymeric substances, and antibacterial agents have difficulty penetrating this barrier and killing and/or inhibiting the proliferation of bacteria within the biofilm. In addition, the biofilm serves as a sanctuary for pathogens, particularly Staphylococcus, Streptococcus, Pseudomonas, and Enterobacter species. Further, the biofilm barrier can also cause encrustation, occlusion, and ultimately failure of the device. As such, methods of preventing and reducing the occurrence and/or severity of biofilm formation, and therefore improving the durability and prolonging the effective life of indwelling medical devices while reducing the risk or morbidity and mortality associated with the use thereof, are continually desired.

In particular, sixty-five percent (65%) of all urinary tract infections are associated with urinary bladder catheterization, and a recent survey found that about nine percent (9%) of health care-associated infections were catheter-associated urinary tract infections (CAUTIs) (Magill et al., N Engl J Med (2014) 370:1198-1208). The species of microorganisms must commonly seen in CAUTIs include Escherichia coli, Enterococcus faecalis, Pseudomonas aeruginosa, and Klebsiella pneumoniae, as well as multiple Candida species; in addition, there are frequently multiple species of microorganisms present in each CAUTI. The exact role of catheter-associated biofilms in CAUTI pathogenesis is poorly understood, but there is evidence that such biofilms play an important role as stable reservoirs for uropathogenic microorganisms that are resistant to antimicrobials and thus difficult to eliminate, even if the catheter is removed (Lehman et al., Antimicrob Agents Chemother (2015) 59:1127-1137).

The use of bacteriophage in controlling biofilm formation has previously been proposed as a natural alternative to antibiotics (see, for example, Lehman et al., op. cit.; Fu et al., Antimicrob Agents Chemother (2010) 54:397-404; and US Patent Application Publication Nos. US 2019/0032022 and US 2009/0191254). However, the use of a single species of bacteriophage does not provide a broad spectrum of protection against the multiple different species of uropathogenic bacteria that can be present in CAUTIs (and also has no effect against any fungal species that may be present). In addition, the covalent attachment of bacteriophage to catheters limits the effectiveness of the bacteriophage to the specific area(s) of the catheter to which the bacteriophage is covalently attached; in other words, only bacteria that are in contact with the exact position on the catheter to which the bacteriophage is covalently attached will be killed. Further, any bacteriophage covalently bound to a surface of a medical implant will be exposed to (and thus subject to inactivation by) the immune system. Yet further, bacteriophage are not very stable at room temperature (thus affecting the storage thereof) and are also subject to degradation at body temperature; as such, covalently bound bacteriophage are typically only stable for about 1-2 days within a patient environment.

Thus, new and improved bacteriophage-containing formulations for disposal on medical devices such as (but not limited to) catheters are desired that overcome the defects and disadvantages of the prior art. It is to such bacteriophage-containing compositions, as well as kits and assemblies containing same and methods of producing and using same, that the present disclosure is directed.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, kits, assemblies, and/or methods disclosed herein can be made and executed without undue experimentation in light of the present disclosure. While the compositions, kits, assemblies, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “polypeptide” as used herein will be understood to refer to a polymer of amino acids. The polymer may include d-, l-, or artificial variants of amino acids. In addition, the term “polypeptide” will be understood to include peptides, proteins, and glycoproteins.

The term “polynucleotide” as used herein will be understood to refer to a polymer of two or more nucleotides. Nucleotides, as used herein, will be understood to include deoxyribose nucleotides and/or ribose nucleotides, as well as artificial variants thereof. The term polynucleotide also includes single-stranded and double-stranded molecules.

The terms “analog” or “variant” as used herein will be understood to refer to a variation of the normal or standard form or the wild-type form of molecules. For polypeptides or polynucleotides, an analog may be a variant (polymorphism), a mutant, and/or a naturally or artificially chemically modified version of the wild-type polynucleotide (including combinations of the above). Such analogs may have higher, full, intermediate, or lower activity than the normal form of the molecule, or no activity at all. Alternatively and/or in addition thereto, for a chemical, an analog may be any structure that has the desired functionalities (including alterations or substitutions in the core moiety), even if comprised of different atoms or isomeric arrangements.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “pharmaceutically-acceptable excipient” refers to any carrier, vehicle, and/or diluent known in the art or otherwise contemplated herein that may improve solubility, deliverability, dispersion, stability, and/or conformational integrity of the compositions disclosed herein.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (i.e., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one tumor and/or cancer. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), certain non-limiting embodiments thereof are directed to an anti-bacterial composition for application to a surface of at least one medical implant and/or for incorporation into a medical implant during the production thereof. The anti-bacterial composition includes a hydrogel that has alginate beads embedded therein; in addition, the alginate beads have a bacteriophage cocktail encapsulated therein. The bacteriophage cocktail comprises: (i) at least one E. coli bacteriophage, and (ii) at least one Klebsiella pneumoniae bacteriophage.

In certain non-limiting embodiments, the bacteriophage cocktail may further comprise one or more additional bacteriophage, such as (but not limited to): the bacteriophage cocktail further comprises at least one of: (iii) at least one Enterococcus faecalis bacteriophage; (iv) at least one Pseudomonas aeruginosa bacteriophage; (v) at least one Staphylococcus aureus bacteriophage; (vi) at least one Staphylococcus saprophyticus bacteriophage; (vii) at least one Proteus mirabilis bacteriophage; and (viii) at least one Streptococcus agalactiae bacteriophage.

In particular (but non-limiting) embodiments, the bacteriophage cocktail includes at least two of (iii)-(viii) (such as, but not limited to, (iii) and (iv)). In other particular (but non-limiting) embodiments, the bacteriophage cocktail comprises three, four, five, or all of (iii)-(viii).

In certain non-limiting embodiments, at least a portion or all of (i)-(viii) is/are a lytic bacteriophage (such as, but not limited to, a Caudovirales lytic bacteriophage). Caudovirales lytic bacteriophage includes three families: Myoviridae, Podoviridae, and Siphoviridae.

In a certain non-limiting embodiment, the bacteriophage cocktail comprises at least four lytic bacteriophage.

In certain non-limiting embodiments, each of the bacteriophage in the cocktail are naturally occurring. In certain non-limiting embodiments, at least one of the bacteriophage in the cocktail is a genetically modified or altered phage.

In another non-limiting embodiment, the bacteriophage cocktail may include (i)-(ii) alone or in combination with one or more of (iii)-(viii) and further in combination with one or more other additional bacteriophage, as described in greater detail herein below.

The non-covalent attachment of the bacteriophage to the medical implant via the hydrogel coating and alginate bead encapsulation provides multiple advantages over the prior art. First, the use of the specific bacteriophage cocktails described herein will target different receptors on the bacteria and provide broad spectrum coverage against up to about 95% to about 99% of the bacteria associated with CAUTIs. Second, the use of the hydrogel and alginate beads preserves the bacteriophage and maintains the stability thereof at room temperature. Third, the alginate beads provide a time release mechanism to the coating, as the beads are stable (i.e., do not melt) at room temperature but will provide gradual release of the bacteriophage as the temperature of the hydrogel increases to body temperature. Fourth, the disposal of the bacteriophage in the hydrogel in a non-covalent manner will allow the bacteriophage to be “free floating” and thus provide greater anti-bacterial protection not only across the entirety of the catheter but also provide anti-bacterial protection to the environment surrounding the catheter. Fifth, encapsulation of the bacteriophage within the alginate beads protects the bacteriophage from the immune system, thereby further increasing the stability and effectiveness thereof. As such, the bacteriophage-hydrogel coating should be stable for at least about 14 days (such as, but not limited to, at least about 30 days); essentially, the bacteriophage-hydrogel coating should be stable and substantially effective for the life of the catheter.

In certain non-limiting embodiments, the anti-bacterial composition is further defined as a time release anti-bacterial composition.

In addition to the bacteriophage listed herein above, in certain non-limiting embodiments, the bacteriophage cocktail may include one or more additional bacteriophage that could be effective against one or more microorganisms associated with UTIs. Non-limiting examples of additional bacteriophages that may be included in the bacteriophage cocktail are T4, LM33-P1, 536_P1, 536_P7, EC200^{PP}, KLPN1, ZCKP1, vB_Klp_5, vB_Klp_2, SRG1, EFD1, and EFLK1, as well as any of the tailed phages Caudovirales, Myoviridae, and Podoviridae.

Alginate is a bio-based polysaccharide extracted from algae that has been widely used as an encapsulation medium for biomedical and pharmaceutical applications because of its favorable properties such as (but not limited to) water solubility, nontoxicity, and biodegradability. Therefore, various types of alginate beads are known in the art as useful as an encapsulation agent. As such, any type of alginate bead known in the art or otherwise contemplated herein for use as a biomedical encapsulation agent can be utilized as a component of the anti-bacterial composition in accordance with the present disclosure.

In certain non-limiting embodiments, the alginate bead is further defined as a sodium alginate bead.

In certain non-limiting embodiments, alginate is present in the alginate bead at any concentration that allows the bead to function in accordance with the present disclosure. For example, alginate may be present in the bead at a concentration of at least about 50%, about 55%, about 60%, about 65%, about 70%, about 75%, about 80%, about 85%, about 90%, about 95%, about 97%, about 98%, about 99%, or higher; also, the alginate concentration may fall within any range of two of the above numbers (i.e., a range of from about 55% to about 75%, a range of from about 60% to about 80%, etc.) as well as any range formed of two integers that each fall between two of the above numbers (i.e., a range of from about 62% to about 76%, a range of from about 58% to about 83%, etc.). Similarly, any other ingredients present in the alginate bead (such as, but not limited to, pectin) may be present in the bead at a concentration of at least about 1%, about 2%, about 3%, about 5%, about 10%, about 15%, about 20%, about 25%, about 30%, about 35%, about 40%, about 45%, or higher; also, the concentration may fall within any range of two of the above numbers (i.e., a range of from about 25% to about 45%, a range of from about 20% to about 40%, etc.) as well as any range formed of two integers that each fall between two of the above numbers (i.e., a range of from about 24% to about 38%, a range of from about 17% to about 42%, etc.).

In particular (but non-limiting) embodiments, the alginate bead comprises alginate and pectin, wherein alginate is present at a concentration in a range of from about 60% to about 80%, and pectin is present at a concentration in a range of from about 20% to about 40%. In a particular (but non-limiting) embodiment, the alginate bead comprises about 70% alginate and about 30% pectin.

In certain non-limiting embodiments, the alginate bead further includes a coating, which further stabilizes the alginate and also increases the stability of the bacteriophage. Non-limiting examples of coatings that may be utilized in accordance with the present disclosure include chitosan and pectin.

The alginate beads may be provided in any form that provides additional stability to the beads as well as the bacteriophage cocktail encapsulated therein. For example (but not by way of limitation), the alginate beads may be embedded within the hydrogel in a substantially dehydrated form and then subsequently substantially rehydrated upon exposure to bodily fluid.

The term “hydrogel” as used herein refers to a colloid in which a solid disperse phase (for example, macromolecules such as collagen, gelatin, agarose, acrylamide, starch, or the like) forms a network in combination with the fluid continuous phase, and wherein the fluid is water. Any hydrogels known in the art or otherwise contemplated herein for in vivo use that are biocompatible and non-immunogenic may be utilized in accordance with the present disclosure.

For example (but not by way of limitation), the hydrogel may be formed of any hydrophilic polymer that allows the anti-bacterial composition to function in accordance with the present disclosure. For example (but not by way of limitation), the hydrogel may be a polyacrylic acid gel, a povidone gel, or a cellulose gel. In addition, the hydrogel may comprise at least one of chitosan, alginate, pectin, agarose, methylcellulose, hyaluronan, collagen, laminin, matrigel, fibronectin, vitronectin, poly-1-lysine, proteoglycans, fibrin glue, gels made by decellularization of engineered and/or natural tissues, as well as any combinations thereof. Further, the hydrogel may comprise at least one of polyglycolic acid (PGA), polylactic acid (PLA), poly-caprolactone (PCL), polyvinyl alcohol (PVA), polyethylene glycol (PEG), methyl methacrylate, poly(methyl methacrylate) (PMMA), poly(2-hydroxyethyl methacrylate) (PolyHEMA), poly(glycerol sebacate), polyurethanes, poly(isopropylacrylamide), poly(N-isopropylacrylamide), or any combination thereof.

In a particular (but non-limiting) embodiment, the hydrogel may contain pectin.

The hydrogel may be provided with any pH that does not damage the portion of a patient with which it comes into contact or cause chemical irritation to the patient upon prolonged exposure to the hydrogel. For example (but not by way of limitation), the gel may have a pH of about 6, about 6.5, about 7, about 7.5, about 8, as well as a range formed from any of the above values (i.e., a range of from about 6 to about 8, a range of from about 6.5 to about 7.5, etc.).

In certain particular (but non-limiting) embodiments, the hydrogel is suspended in a bacteriophage buffer solvent. The bacteriophage buffer solvent may be provided with any formulation known in the art or otherwise contemplated herein that allows the hydrogel to function in accordance with the present disclosure. In a particular (but non-limiting) example, the bacteriophage buffer solvent comprises magnesium.

In certain particular (but non-limiting) embodiments, the anti-bacterial composition further includes at least one additive in addition to the bacteriophage cocktail/alginate beads. Any type of additive that allows the hydrogel to function in accordance with the present disclosure and that may further enhance the properties of the composition may be utilized in accordance with the present disclosure. Non-limiting examples of additives that may be utilized include at least one of an antimicrobial agent, anti-bacterial agent, an anti-viral agent, an anti-fungal agent, a lubricant, or any combinations thereof, and the like.

For example (but not by way of limitation), the anti-bacterial composition may include one or more agents that improve the stability of the coating and/or the effectiveness of the bacteriophage against the CAUTIs. In particular, the CAUTI may include one or more species of Candida; however, bacteriophage are ineffective against yeast. Therefore, the anti-bacterial composition may further include an anti-fungal agent (such as, but not limited to, an anti-Candida agent) to further increase the spectrum of microorganisms against which the anti-bacterial composition is effective.

In another non-limiting example of an additive, the anti-bacterial composition may further include a detectable label that indicates when bacteria are present and encounter the bacteriophage cocktail. For example (but not by way of limitation), the bacteriophage may be modified to express a conjugate of a marker (such as, but not limited to, green fluorescent protein (GFP) attached to a bacteriophage protein; the GFP would then emit a detectable signal as bacteria are being killed.

In certain particular (but non-limiting) embodiments, the anti-bacterial composition has a shelf life of at least about 14 days. For example (but not by way of limitation), the anti-bacterial composition has a shelf life of at least about 30 days.

Certain non-limiting embodiments of the present disclosure are directed to a kit that includes any of the anti-bacterial compositions disclosed or otherwise contemplated herein. The kits may further include at least one medical implant. In certain non-limiting embodiments, the kit includes a pre-assembled medical implant having the anti-bacterial composition pre-applied thereto or incorporated therein; alternatively, the kit includes the anti-bacterial composition packaged separately from the medical implant(s).

Any type of medical implant for which a broad spectrum of protection against biofilm formation is desired can be utilized in accordance with the present disclosure. Non-limiting examples of medical implants include catheters (such as, but not limited to, urinary catheters and intravenous catheters); orthopedic devices (such as, but not limited to, spine screws, rods, and artificial discs; metal or polymer screws, pins, plates, and rods; and the like); endoprosthetic devices (such as, but not limited to, artificial knees, artificial hips, and the like); vascular- and coronary-related implants (such as, but not limited to, coronary stents, implantable cardioverter defibrillators (ICDs), heart pacemakers, and the like), breast implants and other types of augmentation implants; intra-uterine devices (IUDs) and other types of gynecological implants; tympanostomy tubes; intraocular lenses; bone wax; combinations thereof; and the like.

In a particular (but non-limiting) embodiment, the medical implant is a urinary catheter.

The medical implants may be formed of any material and in any manner, so long as the anti-bacterial composition will adhere thereto and/or can be incorporated therein. For example (but not by way of limitation), the medical implant may be formed of a material selected from the group consisting of latex, polyurethane, silicone latex, silicone, vinyl polychloride, and any combination thereof, and the like.

The anti-bacterial composition may be present in the kit in any form that allows the kit to perform in accordance with the present disclosure. For example, but not by way of limitation, the anti-bacterial composition may be provided in a single, individual unit/amount/aliquot, or multiple units/amounts/aliquots of the anti-bacterial composition may be provided within the kit.

In addition to the components described in detail herein above, the kits may further contain other component(s)/reagent(s) for performing any of the particular methods described or otherwise contemplated herein. For example (but not by way of limitation), the kits may additionally include a lubricant and/or an antiseptic agent (such as, but not limited to, povidone iodine, chlorhexidine, octenidine, alcohol, or any combination thereof) to cleanse the patient's skin surrounding the catheter insertion site. In other non-limiting examples, the kit may also include one or more of an antimicrobial agent, an anti-bacterial agent, an anti-viral agent, an anti-fungal agent (such as, but not limited to, an agent effective against Candida species), or any combination thereof, and the like.

The nature of these additional component(s)/reagent(s) will depend upon the particular treatment format and/or area/organ to be treated, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

Each component/reagent present in the kit should be sterile (except for the bacteriophage cocktail).

In addition, the kit can further include a set of written instructions explaining how to use one or more components of the kit. A kit of this nature can be used in any of the methods described or otherwise contemplated herein.

Certain non-limiting embodiments of the present disclosure are directed to an assembly that includes any of the anti-bacterial coating compositions disclosed or otherwise contemplated herein applied to at least a portion of at least one surface of a medical implant (wherein the medical implant is any of the medical implants disclosed or otherwise contemplated herein), or an assembly that includes any of the anti-bacterial compositions disclosed or otherwise contemplated herein incorporated into the formulation from which the medical implant is produced.

When the anti-bacterial coating composition is applied to a medical implant, the hydrogel may be provided with any thickness that allows the gel to function in accordance with the present disclosure. Non-limiting examples of thicknesses that may be utilized in accordance with the present disclosure include about 1 mil, about 5 mil, about 10 mil, about 15 mil, about 20 mil, about 25 mil, about 30 mil, about 35 mil, about 40 mil, about 45 mil, about 50 mil, about 55 mil, about 60 mil, about 65 mil, about 70 mil, about 75 mil, about 80 mil, about 85 mil, about 90 mil, about 95 mil, about 100 mil, or higher, as well as a range that combines any two of the above-referenced values (i.e., a range of from about 10 mil to about 50 mil, etc.), and a range that combines two integers that fall between two of the above-referenced values (i.e., a range of from about 12 mil to about 48 mil, etc.).

Certain non-limiting embodiments of the present disclosure are directed to a method of producing the anti-bacterial composition. The method includes encapsulating any of the bacteriophage cocktails disclosed or otherwise contemplated herein within any of the alginate beads disclosed or otherwise contemplated herein, dehydrating the alginate beads comprising the bacteriophage cocktail, and embedding the dehydrated alginate beads comprising the bacteriophage cocktail within any of the hydrogels disclosed or otherwise contemplated herein.

Methods of encapsulation of materials within alginate beads are well known in the art and are well within the skill of a person having ordinary skill in the art. Thus, no further description thereof is deemed necessary.

Methods of embedding materials within hydrogels are well known in the art and are well within the skill of a person having ordinary skill in the art. Thus, no further description thereof is deemed necessary.

Certain non-limiting embodiments of the present disclosure are directed to a method of preparing a medical implant having an anti-bacterial coating. The method includes the step of applying any of the anti-bacterial coating compositions disclosed or otherwise contemplated herein to at least a portion of a surface of any of the medical implants disclosed or otherwise contemplated herein.

Certain non-limiting embodiments of the present disclosure are directed to a method of preparing a medical implant having an anti-bacterial incorporated therein. The method includes the step of adding any of the anti-bacterial compositions disclosed or otherwise contemplated herein into a formulation and producing any of the medical implants disclosed or otherwise contemplated herein from the formulation.

In a particular (but non-limiting) embodiment, the medical implant is a catheter.

Certain non-limiting embodiments of the present disclosure are directed to a method of reducing the risk of infection associated with placement of a medical implant within a patient. The method includes the step of placing any of the medical implants disclosed or otherwise contemplated herein within a patient, wherein the medical implant has any of the anti-bacterial compositions disclosed or otherwise contemplated herein applied to at least a portion of at least one surface thereof and/or incorporated within the medical implant.

In a particular (but non-limiting) embodiment, the medical implant is a catheter.

Certain non-limiting embodiments of the present disclosure are directed to a method of reducing the occurrence or severity of a urinary tract infection associated with the use of a urinary catheter. The method includes the step of inserting a urinary catheter within a urinary tract of a patient, wherein the urinary catheter has any of the anti-bacterial coating compositions disclosed or otherwise contemplated herein applied to at least a portion of at least one surface thereof.

In certain non-limiting embodiments, the anti-bacterial coating composition is applied to at least a portion of an outer surface of the urinary catheter. Alternatively, and/or in addition thereto, the anti-bacterial coating composition is applied to at least a portion of an inner surface of the urinary catheter.

### Example

An Example is provided hereinbelow. However, the present disclosure is to be understood to not be limited in its application to the specific experimentation, results, and laboratory procedures disclosed herein after. Rather, the Example is simply provided as one of various embodiments and is meant to be exemplary, not exhaustive.

Bacteriophage Propagation:

Fresh, overnight host bacterium that has been streaked out (on plates containing LB agar plus 5 mM CaCl) in order to form isolated colonies was used as the source for the bacteria. 5 mL of sterile LB broth with 5 mM CaCl was then inoculated with a single colony of fresh, overnight bacterium using a sterile inoculation loop, and the inoculated liquid media was placed into a shaking incubator at a speed and a temperature selected based on the host bacteria (i.e., usually at about 200 rpm and about 37° C. for pathogenic host bacteria, but can vary). After incubation for 4-6 hours, 50 mL of bacteriophage stock was added to the bacterial liquid culture, and the mixture was returned to the shaking incubator at the same temperature and rpm and incubated for 12 hours. After 12 hours, the liquid culture was removed and filter sterilized through a 0.2 micrometer filter membrane in order to remove any remaining bacteria. Plaque forming units of the filter sterilized product were then evaluated to determine bacteriophage concentration.

For industrial production, endotoxins need to be removed from the liquid culture using an extra purification step.

Microencapsulation Procedure:

Calcium-alginate microspheres were made using a microencapsulator (such as, but not limited to, Inotech's Encapsulator IER-50 or Encapsulator BIOTECH, Inotech Biosystems International, Inc., Brandon, Fla.). A mixture of high titre bacteriophage stock was suspended in 2% (wt/vol) low-viscosity sodium alginate solution with bacteriophage buffer solvent and was sprayed by the microencapsulator into 50 mM CaCl₂ with a stirring magnet bar. The microspheres were prepared using a 300-600 μm encapsulator nozzle. The fresh microspheres were left to harden in 50 mM CaCl₂ solution for 30 minutes, and then the hardened microspheres were suspended in a solution of chitosan or other natural nonimmunogenic coatings (i.e., chitosan at a 0.4%, wt/vol) for 20 minutes to coat the surface of the alginate microspheres. After coating, the microspheres were rinsed with a sterile water wash and placed in sterile containers stored at 4° C.

Before incorporation into gel coatings, these microspheres were dehydrated either by lyophilization or by methods like serial dehydration in increasing alcohol gradients, depending on bacteriophage sensitivity.

Time Release Pectin-Based Gel:

The time release pectin-based gel can be made with any naturally occurring non-immunogenic water soluble polymer (for example (but not by way of limitation), pectin or agarose). In this pectin example, 4%, wt/vol UVC sterilized pectin powder was combined with sterile Bacteriophage Buffer Solution and homogenized at 90° C. until fully dissolved. Once dissolved, the microencapsulated bacteriophage was then added to the cooling solution 5 minutes after being removed from heat. This produces the finalized coating that is applied to catheters or other medical products.

Bacteriophage Buffer Solution utilized in accordance with the above non-limiting method examples contains 10 ml 1 M Tris, pH 7.5; 10 ml 1 M MgSO₄; 4 g NaCl; and 980 ml dd H₂O. The solution is autoclave sterilized before use.

While the above disclosures describe the inventive concept(s) in conjunction with the specific experimentation, results, and language set forth herein, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. An anti-bacterial coating composition for application to a surface of at least one medical implant, the anti-bacterial coating composition comprising:
a hydrogel;
alginate beads embedded within the hydrogel; and
a bacteriophage cocktail encapsulated within the alginate beads, the bacteriophage cocktail comprising:
(i) at least one E. coli bacteriophage; and
(ii) at least one Klebsiella pneumoniae bacteriophage.

2. The anti-bacterial coating composition of claim 1, wherein (i) and (ii) are Caudovirales lytic bacteriophage.

3. The anti-bacterial coating composition of claim 1, wherein the bacteriophage cocktail further comprises at least one of:
(iii) at least one Enterococcus faecalis bacteriophage;
(iv) at least one Pseudomonas aeruginosa bacteriophage;
(v) at least one Staphylococcus aureus bacteriophage;
(vi) at least one Staphylococcus saprophyticus bacteriophage;
(vii) at least one Proteus mirabilis bacteriophage; and
(viii) at least one Streptococcus agalactiae bacteriophage.

4. The anti-bacterial coating composition of claim 3, wherein the bacteriophage cocktail comprises at least two of (iii)-(viii).

5. The anti-bacterial coating composition of claim 4, wherein the bacteriophage cocktail comprises at least (iii) and (iv).

6. The anti-bacterial coating composition of claim 3, wherein the bacteriophage cocktail comprises three of (iii)-(viii).

7. The anti-bacterial coating composition of claim 3, wherein the bacteriophage cocktail comprises four of (iii)-(viii).

8. The anti-bacterial coating composition of claim 3, wherein the bacteriophage cocktail comprises five of (iii)-(viii).

9. The anti-bacterial coating composition of claim 3, wherein the bacteriophage cocktail comprises (iii)-(viii).

10. The anti-bacterial coating composition of claim 3, wherein at least one of (iii), (iv), (v), (vi), (vii), and (viii) are Caudovirales lytic bacteriophage.

11. The anti-bacterial coating composition of claim 1, further defined as a time release anti-bacterial coating composition.

12. The anti-bacterial coating composition of claim 1, wherein the bacteriophage cocktail further comprises at least one of T4, LM33-P1, 536_P1, 536_P7, and EC200^{PP} bacteriophage.

13. The anti-bacterial coating composition of claim 1, wherein the alginate bead is further defined as a sodium alginate bead.

14. The anti-bacterial coating composition of claim 1, wherein the alginate bead comprises a chitosan coating.

15. The anti-bacterial coating composition of claim 1, wherein the alginate beads embedded within the hydrogel are substantially dehydrated and are substantially rehydrated upon exposure to bodily fluid.

16. The anti-bacterial coating composition of claim 1, further comprising a detectable label that indicates when bacteria are present and encounter the bacteriophage cocktail.

17. The anti-bacterial coating composition of claim 1, further comprising at least one additional anti-microbial agent.

18. The anti-bacterial coating composition of claim 17, wherein the anti-microbial agent is an anti-fungal agent.

19. The anti-bacterial coating composition of claim 1, wherein the hydrogel comprises pectin.

20. The anti-bacterial coating composition of claim 1, wherein the hydrogel is suspended in a bacteriophage buffer solvent.

21. The anti-bacterial coating composition of claim 20, wherein the bacteriophage buffer solvent comprises magnesium.

22. A kit, comprising:
the anti-bacterial coating composition of any one of claims 1-21; and
at least one medical implant.

23. The kit of claim 22, wherein the medical implant is selected from the group consisting of a catheter, an orthopedic device, an endoprosthetic device, a vascular-related implant, a coronary-related implant, a breast implant, an intra-uterine device, a tympanostomy tube, an intraocular lens, and combinations thereof.

24. The kit of claim 23, wherein the medical implant is a urinary catheter.

25. The kit of claim 23, further comprising at least one of a lubricant and an antiseptic agent.

26. An assembly, comprising:
the anti-bacterial coating composition of any one of claims 1-21; and
a medical implant; and
wherein the anti-bacterial coating composition is applied to at least a portion of at least one surface of the medical implant.

27. A method of producing an anti-bacterial composition, the method comprising the steps of:
encapsulating a bacteriophage cocktail within alginate beads, wherein the bacteriophage cocktail comprises:
(i) at least one E. coli bacteriophage; and
(ii) at least one Klebsiella pneumoniae bacteriophage;
dehydrating the alginate beads comprising the bacteriophage cocktail; and
embedding the dehydrated alginate beads comprising the bacteriophage cocktail within a hydrogel.

28. The method of claim 27, wherein the bacteriophage cocktail further comprises at least one of:
(iii) at least one Enterococcus faecalis bacteriophage;
(iv) at least one Pseudomonas aeruginosa bacteriophage;
(v) at least one Staphylococcus aureus bacteriophage;
(vi) at least one Staphylococcus saprophyticus bacteriophage;
(vii) at least one Proteus mirabilis bacteriophage; and
(viii) at least one Streptococcus agalactiae bacteriophage.

29. The method of claim 28, wherein the bacteriophage cocktail comprises two of (iii)-(viii).

30. The method of claim 29, wherein the bacteriophage cocktail comprises (iii) and (iv).

31. The method of claim 28, wherein the bacteriophage cocktail comprises three of (iii)-(viii).

32. The method of claim 28, wherein the bacteriophage cocktail comprises four of (iii)-(viii).

33. The method of claim 28, wherein the bacteriophage cocktail comprises five of (iii)-(viii).

34. The method of claim 28, wherein the bacteriophage cocktail comprises (iii)-(viii).

35. The method of claim 27, wherein at least one of the bacteriophage is a Caudovirales lytic bacteriophage.

36. A method of preparing a medical implant having an anti-bacterial coating, the method comprising the step of:
applying the anti-bacterial coating composition of any one of claims 1-21 to at least a portion of a surface of the medical implant.

37. A method of reducing the risk of infection associated with placement of a medical implant within a patient, the method comprising the step of:
placing a medical implant within the patient, wherein the medical implant has the anti-bacterial coating composition of any one of claims 1-21 applied to at least a portion of at least one surface thereof.

38. A method of reducing the occurrence or severity of a urinary tract infection associated with the use of a urinary catheter, the method comprising the step of:
inserting a urinary catheter within a urinary tract of a patient, wherein the urinary catheter has the anti-bacterial coating composition of any one of claims 1-21 applied to at least a portion of at least one surface thereof.

39. The method of claim 38, wherein the anti-bacterial coating composition is applied to at least a portion of an outer surface of the urinary catheter.

40. The method of claim 38, wherein the anti-bacterial coating composition is applied to at least a portion of an inner surface of the urinary catheter.

41. An assembly, comprising:
a medical implant having an anti-bacterial composition incorporated therein during production thereof, the anti-bacterial composition comprising:
a hydrogel;
alginate beads embedded within the hydrogel; and
a bacteriophage cocktail encapsulated within the alginate beads, the bacteriophage cocktail comprising:
(i) at least one E. coli bacteriophage; and
(ii) at least one Klebsiella pneumoniae bacteriophage.

42. The assembly of claim 41, wherein (i) and (ii) are Caudovirales lytic bacteriophage.

43. The assembly of claim 41, wherein the bacteriophage cocktail further comprises at least one of:
(iii) at least one Enterococcus faecalis bacteriophage;
(iv) at least one Pseudomonas aeruginosa bacteriophage;
(v) at least one Staphylococcus aureus bacteriophage;
(vi) at least one Staphylococcus saprophyticus bacteriophage;
(vii) at least one Proteus mirabilis bacteriophage; and
(viii) at least one Streptococcus agalactiae bacteriophage.

44. The assembly of claim 43, wherein the bacteriophage cocktail comprises at least two of (iii)-(viii).

45. The assembly of claim 44, wherein the bacteriophage cocktail comprises at least (iii) and (iv).

46. The assembly of claim 43, wherein the bacteriophage cocktail comprises three of (iii)-(viii).

47. The assembly of claim 43, wherein the bacteriophage cocktail comprises four of (iii)-(viii).

48. The assembly of claim 43, wherein the bacteriophage cocktail comprises five of (iii)-(viii).

49. The assembly of claim 43, wherein the bacteriophage cocktail comprises (iii)-(viii).

50. The assembly of claim 41, wherein the medical implant is a bone wax.

51. A method of reducing the risk of infection associated with placement of a medical implant within a patient, the method comprising the step of:
placing the assembly of any one of claims 41-50 within the patient.
